(19) United States
(12) Patent Application Publication
(10) Pub. No.: US 2023/0279479 A1
(43) Pub. Date: Sep. 7, 2023

(54) METHODS AND SYSTEMS FOR ANALYZING NUCLEIC ACIDS USING INCREASED IFRET WITH MULTIPLE ACCEPTOR FLUOROPHORES

(71) Applicant: Co-Diagnostics, Inc., Salt Lake City, UT (US)

(72) Inventors: Luming ZHOU, Salt Lake City, UT (US); Carl T. WITTWER, Camden, ME (US); Noriko KUSUKAWA, Camden, ME (US)

(73) Assignee: Co-Diagnostics, Inc., Salt Lake City, UT (US)

(21) Appl. No.: 18/146,051

(22) Filed: Dec. 23, 2022

Related U.S. Application Data
(60) Provisional application No. 63/294,694, filed on Dec. 29, 2021.
(60) Provisional application No. 63/295,388, filed on Dec. 30, 2021.

(51) Int. Cl. C12Q 1/6848; C12Q 1/6818

(52) U.S. Cl. CPC C12Q 1/6848; C12Q 1/6818

(57) ABSTRACT

The present disclosure is directed to methods and processes that may be used to increase the signal of a target-specific reporter molecule (such as a probe) by covalently attaching plural copies of a fluorophore to a target-reporter duplex that are excited by iFRET (induced fluorescence resonance energy transfer) from donor fluorescence of a double-stranded DNA-binding dye bound to the double-stranded DNA structure created by hybridization of reporter and target during an amplification reaction. In one illustrative example, a double-stranded DNA-binding dye is provided in solution and during amplification and/or after completion of amplification, the dye binds to the probe-target duplex, and provides fluorescence resonance energy transfer to multiple acceptor fluorophores that are covalently attached to the duplex.

### CROSS-REFERENCE TO RELATED APPLICATIONS

This application claims the benefit of U.S. Provisional Application No. 63/294,694, filed Dec. 29, 2021 and U.S. Provisional Application No. 63/295,388, filed Dec. 30, 2021, the disclosures of each of which are incorporated herein by reference in their entireties.

### TECHNICAL FIELD

This disclosure relates to systems, methods, and apparatus for detecting and quantifying target nucleic acids, particularly those that are well-suited for detecting target nucleic acids of a particular type that are present in low concentration in a specimen.

### BACKGROUND

The polymerase chain reaction (PCR) has become a method of choice for sensitive and specific detection of pathogens in a sample. The detection of nucleic acids derived from pathogens by PCR is currently the authoritative method for the diagnosis of many infectious diseases. One challenge arising is the detection of target nucleic acids that are present in low concentration in a specimen. Various fluorescent techniques are known and have been used with PCR systems that use sensitive and relatively expensive optical detectors. The expense of such systems and components have been a limit on the ability to develop PCR systems for use outside of a laboratory setting, as for home-based testing.

One such known fluorescent PCR technique is iFRET (induced fluorescence resonance energy transfer). As described in Howell W M, Jobs M, Brookes A L iFRET: an improved fluorescence system for DNA-melting analysis. Genome Res 2002; 12:1401-7, the contents of which are incorporated by reference herein, in this method a solution of a double-stranded DNA-binding dye in the presence of a DNA duplex provides donor fluorescence to an acceptor dye covalently attached to a strand of the duplex. The specific method carried out by Howell used single-labeled probes that hybridized to target nucleic acids immobilized onto a solid support (not in solution). Later, iFRET was shown to work in solution and with asymmetric PCR (Masocj et al., Genotyping by induced fluorescence resonance energy transfer (iFret) Mechanism and Simultaneous Mutation Screening. Human Mutation; Vol 34, No 4 636-643, 2013, the contents of which are incorporated by reference herein) but again only using single-labeled probes. U.S. Pat. No. 6,174,670, the contents of which are incorporated by reference herein in their entirety, discloses methods of monitoring hybridization using fluorescence during PCR, including multiplexing by melting temperature to quantify amplified DNA.

A system or process that has improved signal or signal-to-noise ratio and thus improved sensitivity for detecting target nucleic acids would be an improvement in the art. Such a system that could be used with a device, whether an instrument or receptacle, for monitoring nucleic acid amplification using optical detectors with medium-to-low sensitivity would be a further improvement in the art.

### SUMMARY

The present disclosure is directed to systems, apparatus, and methods and processes that may be used to increase the signal or signal-to-noise ratio of a target-specific reporter molecule using multiple copies of covalently attached fluorophores that are excited by iFRET (induced fluorescence resonance energy transfer) from donor fluorescence of a double-stranded DNA-binding dye bound to the double-stranded DNA structure created by hybridization of a reporter molecule to the target during or after an amplification reaction. Suitable reporter molecule constructs may be formed in various ways that result in plural copies of an acceptor label within the reporter molecule double helix formed by the probe-target duplex.

In a first illustrative example, a double-stranded DNA-binding dye is provided in solution and during amplification and/or after amplification is completed, the dye binds to the probe-target duplex, and provides fluorescence resonance energy transfer to multiple acceptor fluorophores that are covalently attached to the probe-target duplex. In some illustrative examples, the multiple acceptor fluorophores are present on a target specific reporter molecule or probe used in processes in accordance with the present disclosure. In other illustrative examples, multiple acceptor fluorophores are incorporated into the amplified target by use of labeled primers and/or by use of labeled deoxynucleotide triphosphate (dNTP), and said amplified target becomes the reporter molecule. Therefore, when the term reporter molecule” is used in this disclosure, it should be understood to include these many forms of reporter molecules.

### DESCRIPTION OF THE DRAWINGS

It will be appreciated by those of ordinary skill in the art that the various drawings are for illustrative purposes only. The nature of the present disclosure, as well as other embodiments in accordance with this disclosure, may be more clearly understood by reference to the following detailed description, to the appended claims, and to the several drawings.

FIG. 1A shows a schematic of induced fluorescence resonance energy transfer (iFRET) from a double-stranded DNA-binding dye to multiple copies of fluorophore labels on a probe, in the presence of target DNA.

FIG. 1B shows a lack of iFRET in the absence of the target DNA.

FIG. 1C shows exemplary forms of reporter molecule constructs.

FIGS. 2A and 2B show amplification results from the study described in Example 1, where probes labeled with either one carboxy-X-rhodamine (ROX) fluorophore, or two ROX fluorophores were used to detect amplification of the target as a result of fluorescence resonance energy transfer from a double-stranded DNA-binding dye.

FIG. 3A shows melting curves, and FIG. 3B shows negative derivative melting plots of single- and double-labeled probes that were melted from their oligonucleotide complements in the presence of a double-stranded DNA-binding dye as described in Example 2.

FIGS. 4A to 4D show the simultaneous detection of both a SARS-CoV-2 target and human genomic target using probes labeled with two ROX and two Cy5 molecules, respectively, in the presence of a double-stranded DNA-binding dye as the iFRET donor as described in Example 3.

FIG. 5 shows the ratio between acceptor fluorescence intensity (at 640 nm) and donor fluorescence intensity (at 530 nm) at each PCR cycle as described in Example 4. Labels correspond to the primer configuration IDs provided in Table 4 with the number indicating the copy number of ROX acceptor dye.

### DETAILED DESCRIPTION

The present disclosure relates to apparatus, systems, and methods related to detecting and/or quantifying target nucleic acids, particularly those that are well-suited for detecting target nucleic acids of a particular type (e.g., pathogen nucleic acid) that are present in low concentration in a specimen. It will be appreciated by those skilled in the art that the embodiments herein described, while illustrative, are not intended to limit this disclosure or the scope of the appended claims. Those skilled in the art will also understand that various combinations or modifications of the embodiments presented herein can be made without departing from the scope of this disclosure. All such alternate embodiments are within the scope of the present disclosure.

It will be appreciated that while PCR is the amplification method used in the examples of this disclosure that it is understood that any nucleic acid amplification method compatible with the use of double-stranded DNA-binding dye may be used for detection and/or quantification using the methods and processes disclosed herein. It will be appreciated that while PCR is the amplification method used in the examples herein, it is understood that any amplification method that uses a primer may be suitable, whether the signal or target is amplified. In fact, any proximity-based amplification approaches known to those of skill in that art may be used, including assays for the signal amplification to detect antigens. Some suitable procedures may include polymerase chain reaction (PCR); strand displacement amplification (SDA); nucleic acid sequence-based amplification (NASBA); cascade rolling circle amplification (CRCA), loop-mediated isothermal amplification of DNA (LAMP); isothermal and chimeric primer-initiated amplification of nucleic acids (ICAN); target based-helicase dependent amplification (HDA); transcription-mediated amplification (TMA), CRISPR-Cas9-triggered strand displacement amplification, immuno-PCR, recombinase polymerase assay (RPA) and the like. Amplification methods may include pre-enrichment steps such as antibody- or affinity-mediated capture or precipitation of microorganisms, other means to concentrate the target microorganism, and pre-enrichment of the target nucleic acid sequence such as by immobilized probes, by whole genome amplification, by nested PCR, or the like. Amplification methods may further include analyses such as melting curve analysis, high-resolution melting, and high-speed melting analyses. Therefore, when the term PCR is used, it should be understood to include other alternative amplification methods, amplification that is preceded by enrichment, and analysis of amplification products. It is understood that protocols may need to be adjusted accordingly.

The present disclosure is directed to methods for detecting and/or quantifying target nucleic acids that have general application, but that are particularly well-suited for detecting target nucleic acids of a particular type (e.g., pathogen nucleic acid) that are present in low concentration in a specimen. This also opens an opportunity to build instruments for monitoring nucleic acid amplification using optical systems that are less expensive and less sensitive than those commonly found in PCR devices that typically use expensive discrete lenses, interference filters and multiple detectors.

In particular, methods and processes in accordance with the present disclosure may be used to increase the signal or signal-to-noise ratio of a target-specific reporter molecule using multiple copies of covalently attached fluorophores that are excited by iFRET (induced fluorescence resonance energy transfer) from donor fluorescence of a double-stranded DNA-binding dye bound to the double-stranded DNA structure created by hybridization of a reporter molecule to the target during or after a nucleic acid amplification reaction. Suitable reporter molecule constructs may be formed in various ways that result in plural copies of an acceptor label within the reporter molecule double helix formed by the reporter-target duplex.

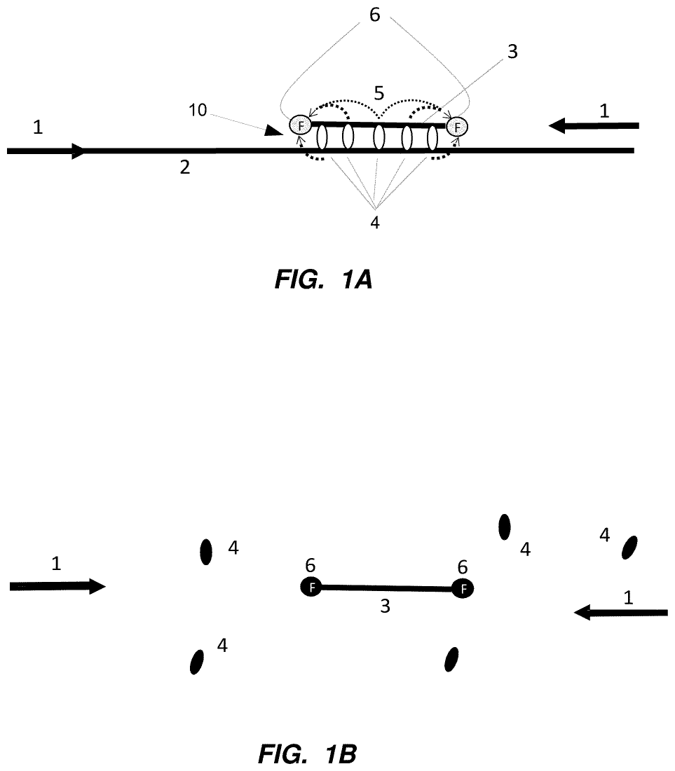

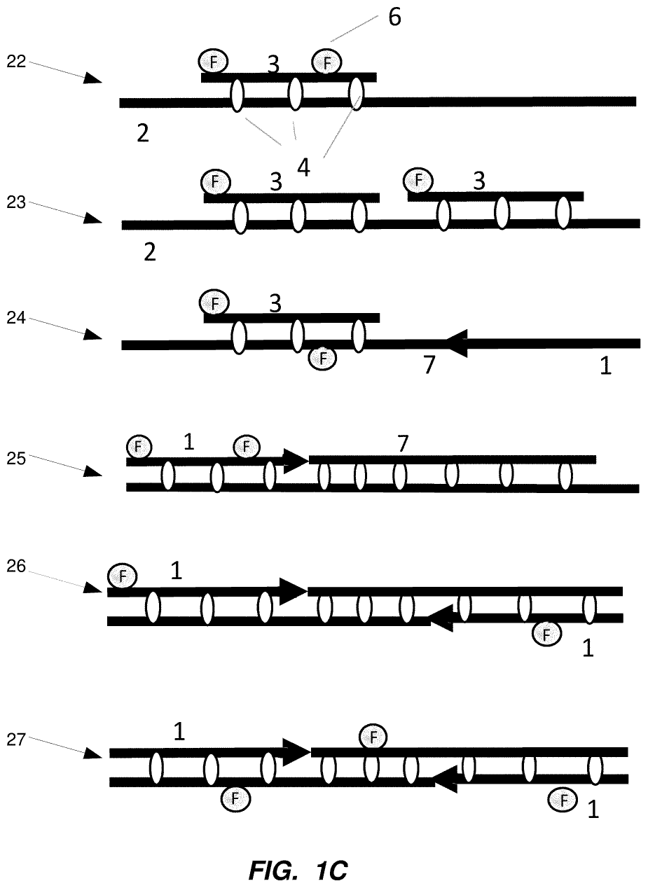

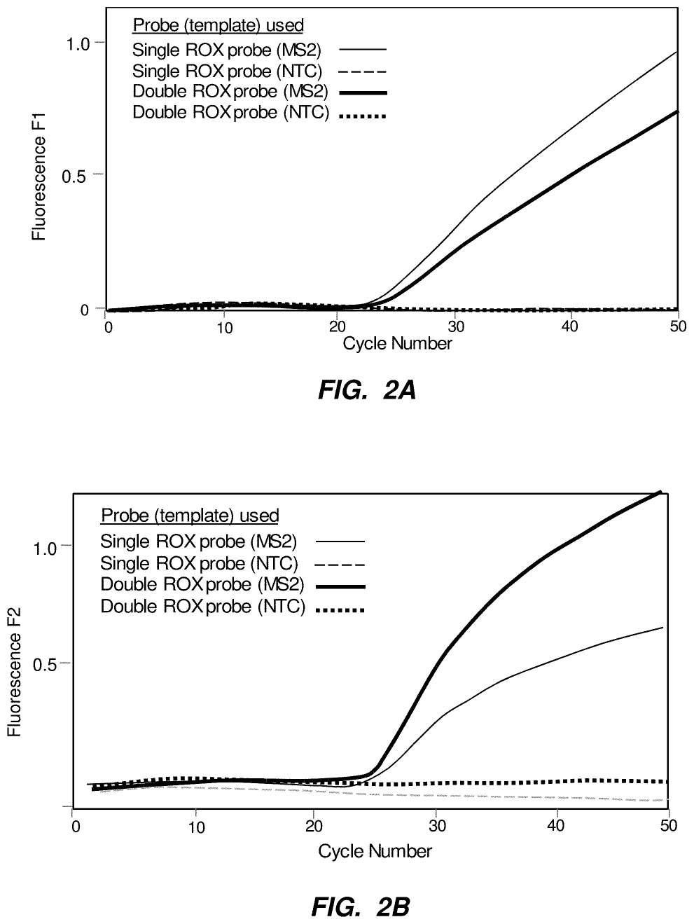

A first illustrative example is depicted in FIGS. 1A and 1B in which a reporter molecule is a probe with multiple acceptor labels.

FIG. 1A shows a schematic of iFRET from a double-stranded DNA-binding dye to multiple copies of fluorophores labels on a probe 3, in the presence of target DNA 2. The double-stranded DNA-binding dye 4 is provided in solution. During annealing, the dye binds to the probe-target duplex, generally indicated at 10, and will provide fluorescence resonance energy transfer 5 to the acceptor fluorophores 6 that are covalently attached to the probe 3 that forms the duplex 10 with the target DNA 2. As depicted in FIG. 1B, primers 1 are unable to initiate amplification in the absence of the target, and there will be no probe-target duplex, thus the double-stranded DNA-binding dye 4 will not fluoresce, and the labels 6 on the probe 3 will not excite, staying dark. The double-stranded DNA-binding dye and fluorescent labels on the probe may be selected so that the double-stranded DNA binding dye can be efficiently excited at wavelengths that do not directly excite the fluorescent labels.

It will be appreciated that a reporter molecule includes various constructs as exemplified in FIG. 1C, with the requirement being that, collectively, there are plural copies of an acceptor label within the double helix formed by the reporter molecule. Therefore, a reporter molecule can be a probe 3 with multiple acceptor labels 6, as depicted at 22, or multiple probes each with at least one label, as depicted at 23. It can also be a probe-target duplex in which the probe may or may not be labeled provided the target is an extension product 7 labeled by incorporation of a labeled dNTP (plural copies, if probe is not labeled), as depicted at 24. A reporter molecular can further be an extension product of a primer with multiple labels, as depicted at 25, a duplex of extension products (also called an amplicon or a PCR product) with multiple labels incorporated during amplification either by a pair of labeled primers, as depicted at 26, or by a plurality of a labeled dNTP, or by a combination of at least one labeled dNTP and a primer with at least one label as depicted at 27. Therefore, when the term “probe” or “reporter molecule” is used in this disclosure, it should be understood to include these many forms of reporter molecules in the target duplex.

A target nucleic acid sequence can originate from an organism of interest or a variant/mutation of interest, or can also be a tag or barcode nucleic acid sequence which is released during specific amplification to which the reporter molecule hybridizes.

By increasing the copy number of acceptor fluorophores, not only is the signal of the reporter molecule increased, but background from the double-stranded DNA binding dye is decreased, resulting in improved signal-to-noise ratio in the optical channel in which the acceptor signal is primarily detected but also may see spill-over signal from the donor dye. Examples of such decrease in dye signal by increase in acceptor fluorophore copy number are shown in Examples 2 and 3.

Commonly, spill-over, or crosstalk, between different fluorophores is algorithmically solved by color compensation methods (e.g., as in U.S. Pat. No. 6,197,520, the contents of which are incorporated by reference herein in its entirety). However, for targets that are present in low concentrations (such as virus RNA or DNA), particularly in the presence of higher concentrations of background nucleic acids (such as human DNA) or when used with low-sensitivity detectors, color compensation is expected to be less effective, and decreasing the interfering signal will be helpful.

Double-stranded DNA-binding dyes, are dyes that have very little fluorescence when free, but emit a strong signal when bound to double-stranded DNA. Non-limiting examples of such dyes include SYBR Green I (ThermoFisher Scientific Corporation, Carlsbad, Calif.), EvaGreen (Biotium, Fremont, Calif.), LC Green Plus (BioMerieux, Salt Lake City, Utah), SYTO 9, SYTO 40 (both from Thermo Fisher Scientific, Waltham, Mass.), and Maverick Blue (Co-Diagnostics, Inc., Salt Lake City, Utah).

Detection of target can be accomplished either by quantitative or qualitative real-time PCR (for DNA targets) or real-time RT-PCR (for RNA targets), and/or melting curve analysis of the probe-target hybrid and/or that of the PCR product (amplicon). In an illustrative example, a probe tagged with more than one acceptor fluorescent label is added to a PCR mixture containing a double-stranded DNA-binding dye. Illustrative configurations of such probes are shown in Table 1.

TABLE 1
Possible configurations of multi-labeled probes

| | Probe description | Configuration | Schematic (F is the fluorescent label) |
|---|---|---|---|
| A | Dual labeled | Label on 5′- and 3′- ends of an oligonucleotide | 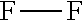 |
| B | Dual labeled | One label on 5′ or 3′ end with a second internal label affixed through a linker (e.g., amino- | 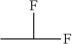 |
| | | modifier C2 or C6 dT) | |
| | | | 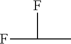 |
| C | Triple labeled | One internal label, and two labels on the ends | 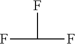 |
| D | Quadruple labeled | Two internal labels and one each on each end. | 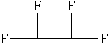 |
| E | Higher-order labeling | Additional internal labels added by incorporating additional amino linkers | 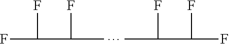 |

To increase FRET, the distance between the donor dye and receptor fluorophore should be minimized, and therefore, the linkers on the amino-modifiers should be short, e.g., a two-carbon (C2) to a six-carbon (C6) linker. In general, double-stranded DNA-binding dyes incorporate one dye molecule every 4 to 10 base pairs, so multiple locations of donor emissions are available along the double-stranded DNA. When probes or primers are labeled with multiple acceptor fluorophores, the best acceptor fluorophore spacing is a compromise between (1) increased fluorescence resulting from more than one copy of acceptor fluorophore, and (2) decreased fluorescence from quenching between acceptor fluorophores. The distance between acceptor fluorophores on the same DNA strand is a function of both the number of bases between the fluorophores and their relative radial position around the double helix. Quenching can be minimized (without affecting the average donor-to-acceptor distance) by placing acceptor fluorophores on opposite sides of the DNA helix, which completes one turn in 10.5 bases. Therefore, based on radial position, optimal spacing would be 5.25, 15.75, and 26.25 bases, or 4.25, 14.75 and 25.25 unlabeled bases between adjacent labeled bases. However, quenching is likely too strong with the shortest spacing of 4 bases, and the longest spacing of 25 bases will leave some donor dyes unutilized. Therefore, a spacing of 15 bases is geometrically optimal for multiple iFRET labeling. Acceptor fluorophores with an 8-23 base separation are preferred, with a 10-20 base separation more preferred, 13-17 base separation even more preferred, and a 15 base separation is most preferred.

Fluorophores may be added during oligonucleotide synthesis on the 3′-end with a labeled CPG support, and on the 5′-end with labeled phosphoramidites, or by post-synthesis on amino-linkers through N-hydroxysuccinimide (NHS) ester coupling as is known in the art. Multiple identical labels can be added simultaneously onto multiple amino linkers on the same probe through NHS ester coupling. Internal fluorophore labeling on amino-linkers attached to DNA bases are preferred so as to minimize effects on hybridization, specifically amino-modifier C6dT, as well as amino-modifier C6dA, C6dC, and C6dG (Glen Research, Sterling, Va.).

Fluorophores can also be incorporated into the reporter molecule by use of a fluorescent dNTP optionally mixed with its non-fluorescent counterpart in the amplification reaction mixture. The nucleic acid sequence of the amplified reporter molecule and/or the ratio of labeled to nonlabelled dNTP determine the actual or average frequency of incorporation or spacing of fluorescent labels along the length of the amplicon. Nonlimiting examples of a labeled dNTP are rhodamine-12-dUTP, dCTP-Cy5, dUTP-Texas Red, dCTP-Cy3, or fluorescein-12-dUTP (Jena Bioscience, Jena, Germany).

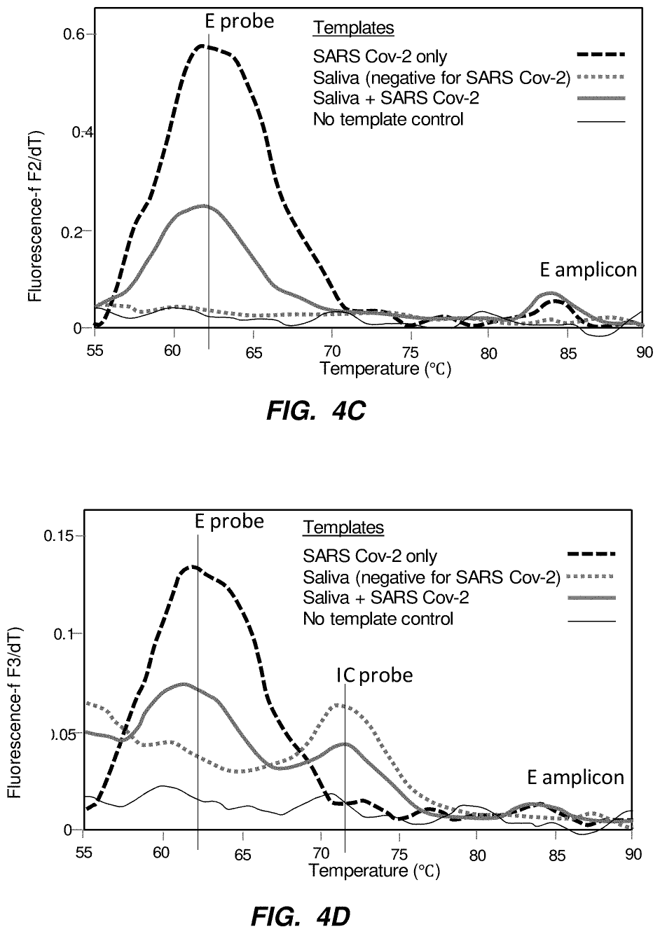

The amplification reaction mixture may be illuminated at the excitation wavelength of the double-stranded DNA binding dye, and detection may be performed using the wavelength of emission of the reporter label(s). It is often not possible, nor is it necessary, to exactly match the illumination of dye at its peak excitation wavelength as long as the dye can be excited. Similarly, it is not necessary to detect the signal of the reporter label at its peak emission wavelength. If the emission spectra of two or more reporter labels are sufficiently spaced apart, multiple targets can be detected simultaneously using different colors with this method (FIGS. 4C and 4D). Illustrative combinations are shown in Table 2.

TABLE 2
Illustrative combination of dyes and labels

| | | | Excitation source | |
|---|---|---|---|---|
| | dsDNA-binding | | wavelength (LED or | Detect |
| ID | dye (donor) | Labels (acceptor/s) | laser diodes) | approximately at |
| A | Maverick Blue | Yakima Yellow dual label | 450 nm | ≥575 nm |
| B | Maverick Blue | ROX dual label | 450 nm | ≥600 nm |
| C | Maverick Blue | Cal Fluor Red 610 dual label | 450 nm | ≥600 nm |
| D | Maverick Blue | Cy5 dual label | 450 nm | ≥650 nm |
| E | Maverick Blue | HEX dual label | 450 nm | ≥575 nm |
| F | Maverick Blue | Alexa 660 dual label | 450 nm | ≥600 nm |
| G | Maverick Blue | ROX dual label (target 1) | 450 nm | 630 nm (target 1) |
| | | Cy5 dual label (target 2) | | 680 nm (target 2) |
| H | Maverick Blue | ROX dual label (target 1) | 450 nm | 630 nm (target 1) |
| | | Alexa 660 dual label (target 2) | | 680 nm (target 2) |
| I | LC Green Plus | LCRed640 dual label | 470 nm | ≥600 nm |
| J | EvaGreen | ROX dual label | 488 nm | ≥600 nm |
| K | SYTO40 | JOE dual label (target 1) | 405 nm | 555 nm (target 1) |
| | | ROX dual label (target 2) | | 630 nm (target 2) |
| | | Alexa 660 dual label (target 3) | | 690 nm (target 3) |
| L | SYBR Green I | Cy5 dual label | 488 nm | ≥650 nm |
| M | SYBR Green I | Alexa 660 dual label | 488 nm | ≥680 nm |

It will be appreciated that prior to the present disclosure it appears that affixing more than one copy of a fluorescent label onto a probe or primer is seldom done, and has not been used in combination with iFRET. A typical probe or primer is 15-30 nucleotides long, and thus has limited space available for multiple labels to be covalently affixed to it. The common concern with using multiple copies of labels in such proximity is the possibility of intramolecular quenching which will dampen the signal rather than increase the signal. In fact, in protein analysis, the dampening of signal by bringing two identical labels close to each other is used to study clustering of identical proteins (see e.g., Edwin et al., Enumeration of Oligomerization States of Membrane Proteins in Living Cells by Homo-FRET Spectroscopy and Microscopy: Theory and Application; Biophysical Journal 92(9) 3098-3104, 2007, the contents of which are incorporated by reference herein in its entirety). Applicant has found that iFRET procedures using double-stranded DNA-binding dye to multiple copies of acceptor fluorophore labels on a probe-target duplex or on an amplicon in accordance with the present disclosure do not suffer from such self-quenching as long as the number of unlabeled bases between adjacent fluorophores is at least 8 bases. A spacing between acceptor fluorophores of about 15 bases (or equivalent distances using linkers) is optimal. Longer spacing is also useful in increasing acceptor signal although it will leave some of the donor fluorescence unused for iFRET. Therefore, with long reporter molecules, the preferred approach is to tile acceptor fluorophores every 10 to 20 bases along the available length (to the extent that such tiling does not impede performance of the reporter molecule) so that iFRET is maximized and background signal from the donor dye is minimized. Without being limited to a particular mechanism, it is surmised that the accumulation of energy from a chain of donor dye molecules into multiple acceptor fluorophores overcomes any quenching between them. Advances in oligonucleotide synthesis and purification have also lowered the cost of providing multiple labels, enabling the multiple label approach.

It will be appreciated that methods and processes in accordance with the present disclosure may include additional steps to further increase the signal of the reporter probe, such as by use of asymmetric PCR to favor the production of the target DNA strand that hybridizes to the probe, thus reducing competition from its complementary DNA strand, as is known in the art. When labeled primers are used, keeping the amplicon short will limit donor fluorescence that is not transferred to the acceptors while effectively increasing the acceptor fluorescence by use of multiple acceptor dyes. An ideal configuration is to have both primers labeled at their 5′ ends and at one internal position about 5 bases from their 3′ ends (so as not to impede extension), with a 5 base pair separation between primers. Assuming 20 base primer pairs, such configuration incorporates 4 acceptors into a 45 base amplicon all at 15 base spacing, maximizing the acceptor fluorescence from iFRET and minimizing residual donor fluorescence. Another additional step to increase the signal of the reporter probe is to have two, or more probes that specifically hybridize to a region different from the first probe but on the same gene or on the same genome, all labeled with a plurality of the same fluorophore as the first probe. If melting analysis is to be used, then all probes for the same gene or same genome may be designed to have equivalent melting temperatures. Further, the detection of targets can be multiplexed optionally by use of their differences in melting temperature (Tm) as is known in the art. This is particularly useful when color compensation, and reduction of the double-stranded DNA binding dye signal is not enough to fully discriminate two or more targets that are hybridized to their respective probes that have crosstalk despite being labeled with different-colored fluorophores.

### EXAMPLES

### Example 1

Amplification Curves with One or Two Copies of Fluorophore on an iFRET Probe.

A 73 bp fragment of the single-stranded RNA bacteriophage MS2 was amplified with forward primer TCCAGGGTGCATATGAG SEQ ID NO. 1 (Tm=59.41° C.) and reverse primer TTAGTACCGACCTGACG SEQ ID NO. 2 (Tm=59.58° C.) in the presence of either the single-labeled ROX probe AAGAGTTTCTTCCTATGAGAGCC-ROX SEQ ID NO. 3 or the double-labeled ROX probe: ROX-AAGAGTTTCTTCCTATGAGAGCC-ROX SEQ ID NO. 3 (Tms=64.06° C.), all obtained from IDT (Coralville, Iowa). The reaction mixture included 2×10⁴ copies of MS2, 0.25 μM limiting forward primer, 0.5 μM reverse primer, 0.5 μM labeled probe, 200 μM of each dNTP (Sigma-Aldrich, St. Louis, Mo.), 20 μM Maverick Blue nucleic acid stain (Idaho Molecular, Inc., Salt Lake City, Utah), 3.2 U/μL GoScript reverse transcriptase (ProMega, Madison, Wis.), 0.04 U/μL KlenTaq 1 (DNA Polymerase Technologies, St Louis, Mo.), 4 mM MgCl₂ (Sigma), 125 μg/mL bovine serum albumin (Sigma), and 50 mM Tris, pH 8.3 in a 10 μL LightCycler capillary (Roche Molecular Systems, Indianapolis, Ind.). Samples were temperature cycled on a capillary LightCycler with a reverse transcription step of 45° C. for 30 s, followed by denaturation at 95° C. for 30 s, and then 50 cycles of amplification between 95° C. for 0 s and 55° C. for 0 s. Fluorescence was collected at 55° C. each cycle in the F1 (530+/−20 nm) and F2 (640+/−30 nm) channels.

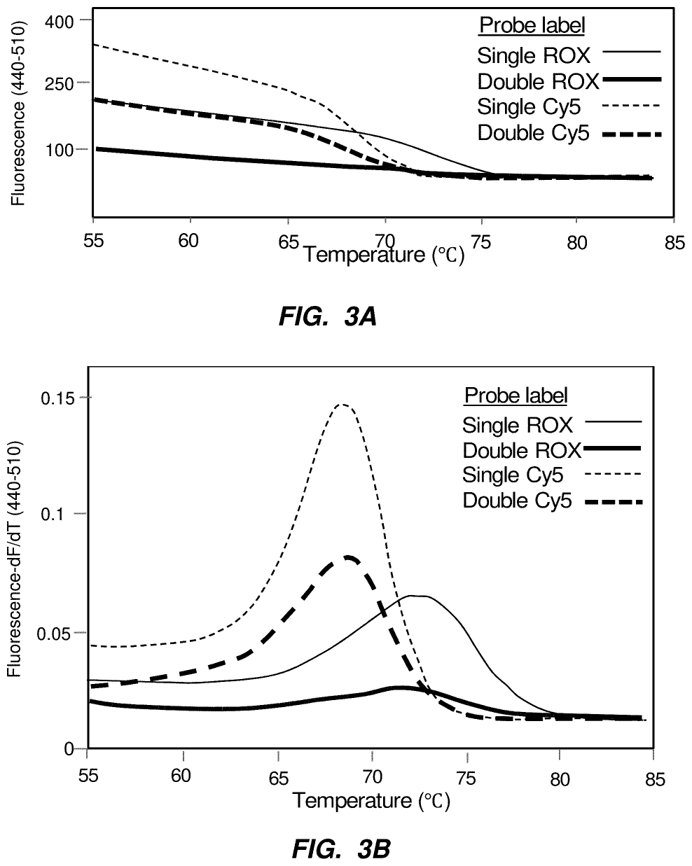

The results are depicted in FIGS. 2A and 2B. FIG. 2A shows the amplification curve of the MS2 target as observed in the F1 channel capturing the emission of Maverick Blue (471 nm) in which the higher dose of fluorophore on the probe decreased the Maverick Blue signal. FIG. 2B shows the same amplification curve observed at a longer wavelength (F2) more suited for observing the signal of ROX (emission peak at 604 nm). Here, the higher dose of ROX on the probe provided a higher signal. In fact, doubling the number of ROX roughly doubled its signal. These results thus demonstrate that: (A) the Maverick Blue fluorescence in F1 is lower in the presence of the double-labeled probe compared to the single-labeled probe, and (B) The ROX fluorescence in F2 is greater for the double-labeled probe compared to the single-labeled probe. The no-template controls (NTC) showed no amplification.

### Example 2

Melting Peak Signals with One or Two Copies of Fluorophore on iFRET Probes.

Single- and double-labeled probes were melted from their reverse complements. MS2 single- and double-labeled probes were of the same sequence given in Example 1 but were labeled with Cy5. In addition, single- and double-labeled probes targeting the human RNase P gene were AAGGCTCTGCGCGGACTTG-ROX SEQ ID NO. 4 and ROX-AAGGCTCTGCGCGGACTTG-ROX SEQ ID NO. 4. Probes and their reverse complements were mixed at equimolar concentrations (0.5 μM each) in the presence of 20 μM Maverick Blue, 3 mM MgCl₂, 125 μg/ml BSA and 50 mM Tris, pH 8.3. Twenty microliter aliquots of each probe/complement combination were melted in a LightCycler 480 instrument from 50 to 85° C. at a rate of 5 acquisitions/° C. Excitation was at 440 nm with emission monitored at 488, 510, 580, 610, 640, and 660 nm.

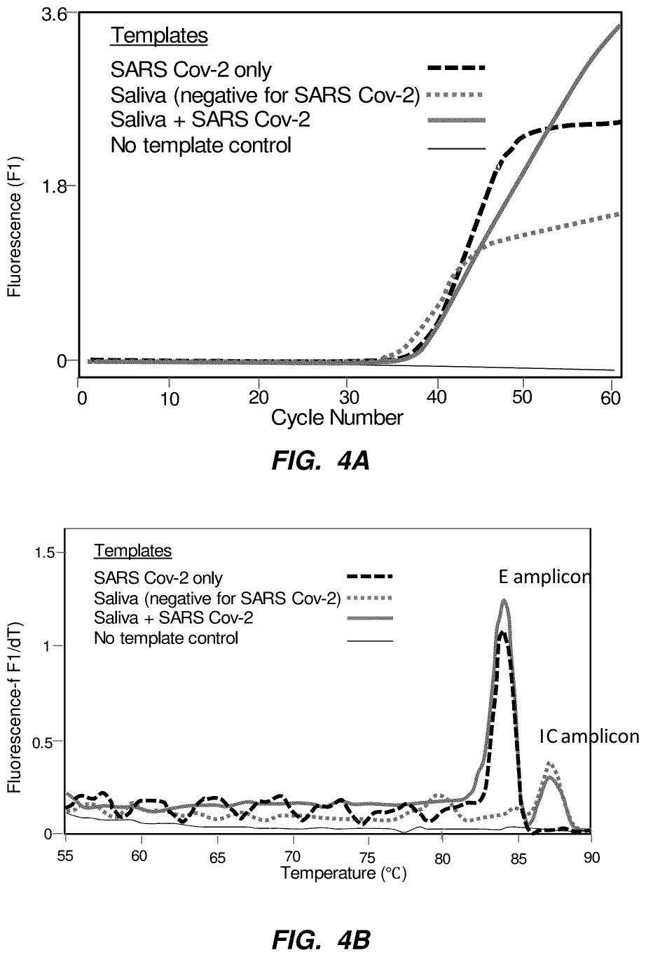

FIG. 3A and FIG. 3B show the results of 510 nm emission where only the signal of Maverick Blue dye is observed. FIG. 3A shows melting curves, and FIG. 3B shows negative derivative melting plots of single- and double-labeled probes that were melted from their oligonucleotide complements in the presence of Maverick Blue. The four curves display melting of single- and double-labeled probes directed towards fragments on two different targets, the bacteriophage MS2 and the human gene RNase P. For both the Cy5-labeled probes and the ROX-labeled probes, the Maverick Blue signal is decreased for double-labeled probes compared to single-labeled probes. That is, double-labeled probes more effectively collect the emitted light from Maverick Blue as compared to single labeled probes. The enhanced conversion to longer wavelengths from double-labeled probes can be seen in Table 3 where the relative peak heights of all emission channels are listed.

TABLE 3
Comparison of relative peak heights on derivative
melting plots (excited at 440 nm)

| | | | Approximate wavelength used for detection | | | | | |
|---|---|---|---|---|---|---|---|---|
| | | Dose of | 488 | 510 | 580 | 610 | 640 | 660 |
| Probe | Label | label | nm | nm | nm | nm | nm | nm |
| MS2 | Cy5 | Single | 65 | 36 | 3 | 1.1 | 4.3 | 30 |
| MS2 | Cy5 | Double | 34 | 19 | 1.4 | 0.7 | 5.5 | 45 |
| RNase P | ROX | Single | 25 | 14 | 15 | 61 | 27 | 22 |
| RNase P | ROX | Double | 7 | 4 | 22 | 103 | 46 | 38 |

At lower wavelengths that monitor Maverick Blue emission (488 and 510 nm), double-labeled probes emit only 28-53% of the light of single-labeled probes. Whereas, at higher wavelengths (610 and 640 nm for ROX, 640 and 660 nm for Cy5), double-labeled probes emit 127-170% of the light of single-labeled probes. This suppression of donor fluorescence and augmentation of acceptor fluorescence is expected to increase further when triple, quadruple, or even more labels are added to iFRET probes.

### Example 3

Multiplexing iFRET with Double-Labeled Probes Using Both Color and Probe Tm.

The SARS-CoV-2 E gene and the human internal control gene RNase P were amplified simultaneously in the presence of double-labeled probes that differed both in emission color and probe melting temperatures. A 122 bp product of the E gene was amplified with forward primer TTCGGAAGAGACAGGTACGTTA SEQ ID NO. 5 and reverse primer TATTGCAGCAGTACGCACA SEQ ID NO. 6 with probe ROX-ACTAGCCATCCTTACTGCa-ROX SEQ ID NO. 7 where “a” is a non-complementary base added to limit fluorophore quenching from GC base pairs. A 72 bp product of the RNase P gene was amplified with forward primer GCGGTGTTTGCAIATTTIG SEQ ID NO. 8 where I (inosine) is substituted for G to lower the primer Tm. The RNase P reverse primer was GGCTGTCTCCACAAGTC SEQ ID NO. 9 and RNase P probe was Cy5-AAGGCTCTGCGCGGACTT-Cy5 SEQ ID NO. 10. Templates for amplification variably included 2×10³ copies of heat-inactivated SARS-CoV-2 (VR-1986) (ATCC, Manassas, Va.), 2 μL human saliva as the source of human DNA, both or neither (no template control) in a 10 μL reaction. The concentrations of E gene primers were 0.25 μM forward and 0.5 μM reverse, and RNase P primers were 0.125 μM forward and 0.25 μM reverse. The double-labeled E gene ROX probe was at 0.5 μM and the double-labeled RNase P gene Cy5 probe at 0.25 μM. Enzymes, dNTPs and buffer components were the same as in Example 1. Samples were temperature cycled on a capillary LightCycler with a reverse transcription step of 45° C. for 30 s, followed by denaturation at 95° C. for 30 s, and then 60 cycles of 3-step amplification at 95° C. for 0 s and 55° C. for 0 s and 76° C. for 5 s. Fluorescence was collected at 55° C. each cycle in the F1 (530+/−20 nm) channel and displayed in FIG. 4A. After amplification, melting analysis was performed by heating to 95° C., cooling to 50° C., and then heating at 0.3° C./s to 95° C. with continuous fluorescence acquisition.

FIG. 4A shows amplification curves as observed at the wavelength of Maverick Blue emission. FIG. 4B shows the derivative melting plots at the wavelength of Maverick Blue in which the melting peaks of the SARS-Cov-2 amplicon (E amplicon) and the human genomic target amplicon (IC amplicon) were visible. FIG. 4C shows the derivative melting plots observed at the wavelength for ROX emission in which the melting peaks for the SARS-Cov2 probe (E probe) and amplicon (E amplicon) were visible. FIG. 4D shows the derivative melting plots observed at the wavelength of Cy5 emission in which the melting peaks for SARS-Cov2 probe (E probe) and amplicon (E amplicon), and the human genomic probe (IC probe) were all visible.

Derivative melting curves for channels F1, F2 and F3 show Maverick Blue fluorescence of both target amplicons (FIG. 4B), E-gene ROX fluorescence of probe (FIG. 4C), and both the E-gene ROX fluorescence and the RNase P Cy5 fluorescence of probes (FIG. 4D), respectively. In FIG. 4A, when SARS-CoV-2 RNA and/or human DNA (saliva) were present amplification occurred and Maverick Blue detected the amplified dsDNA with a quantification cycle (Cq) of about 35. The negative control without template showed no amplification. The two target amplicons can be easily distinguished on negative derivative melting curve plots in channel F1 (FIG. 4B). The E amplicon melted at about 84° C. while the human DNA internal control (IC) amplicon melted at about 87° C. In channel F2 (FIG. 4C), although there were small residual amplicon peaks from the fluorescence tail of Maverick Blue, the only fluorescence apparent in the probe region was from the SARS-CoV-2 E gene labeled with ROX with a Tm of about 62° C. The saliva sample that only contained human DNA showed no peak in F2. However, the saliva sample did show a melting peak in F3 (FIG. 4D, IC probe) with a Tm of 72° C. from the Cy5 labeled probe for the human internal control gene RNase P. The sample with only SARS-CoV-2, although present in F3 had a Tm of 62° C. and was easily distinguished from the Cy5 probe at 72° C. In summary, both color (different fluorescent labels as acceptor dyes on different labeled iFRET probes) and Tm (melting temperature differences between probes) can be used for multiplexing targets. When probes differ in both color and Tm, the extra assurance of target identity allows higher levels of multiplexing by using both color and Tm as distinct differentiation axes.

### Example 4

iFRET with Zero to Four Copies of Acceptor Fluorophore

A short PCR product was used to demonstrate fluorescence energy transfer from Maverick Blue dye (donor) to zero, one, two, three or four copies of ROX fluorophore (acceptor). The PCR product was generated by use of the forward and reverse primer configurations in Table 4.

TABLE 4
Primer configurations to generate labeled amplicons

| | Copy number of | Forward primer (left) and reverse primer (right) |
|---|---|---|
| ID | fluorescent label | configurations (F is the fluorescent label) |
| 0 | 0 | 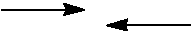 |
| 1A | 1 | 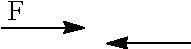 |
| 1B | 1 | 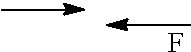 |
| 2A | 2 | 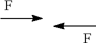 |
| 2B | 2 | 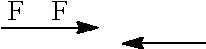 |
| 2C | 2 | 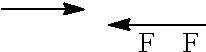 |
| 3A | 3 | 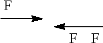 |
| 3B | 3 | 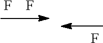 |
| 4 | 4 | 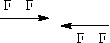 |

Forward primer TTAAACCAGGTGGAACC SEQ ID NO. 11 and reverse primer AGTTGTGGCATCTCCT SEQ ID NO. 12 were used to amplify a short 5 bp sequence between the primers, resulting in an amplicon of 38 bp with the sequence TTAAACCAGGTGGAACCtcatcAGGAGATGCCACAACT SEQ ID NO. 13 (the 5 bp sequence is shown in lower case). Donor fluorophore ROX could be attached to the primers at the 5′ terminus and/or the underlined thymine bases. When ROX was attached to all four possible positions, spacings between fluorophores on the resulting amplicon were 10, 16, and 10 bases (not counting the thymine bases to where ROX was attached). PCR was performed with 0.25 μM of each primer pair, 20 μM Maverick Blue nucleic acid stain, 10⁴ copies/μL of a synthetic double-stranded DNA template with the same sequence as the amplicon, 0.04 U/μL KlenTaq 1 DNA polymerase, 200 μM of each dNTP, 4 mM MgCl₂, 125 μg/mL bovine serum albumin, and 50 mM Tris, pH 8.3 in a 10 μL sample volume. Samples were temperature cycled using a LightScanner32 real-time PCR instrument (Idaho Technology, Inc., Salt Lake City, Utah) with 45 cycles of 3-step amplification at 95° C. for 5 s, 50° C. for 10 s, and 72° C. for 5 s using ramp rates of 20° C./s. Fluorescence intensity data were collected at 50° C. each cycle in the 530 nm (Maverick Blue) and the 640 nm (ROX) channels. Compared to the single-labeled iFRET configuration (1A, 1B in Table 4), a clear increase in the ROX signal was achieved by increasing the number of ROX labels.

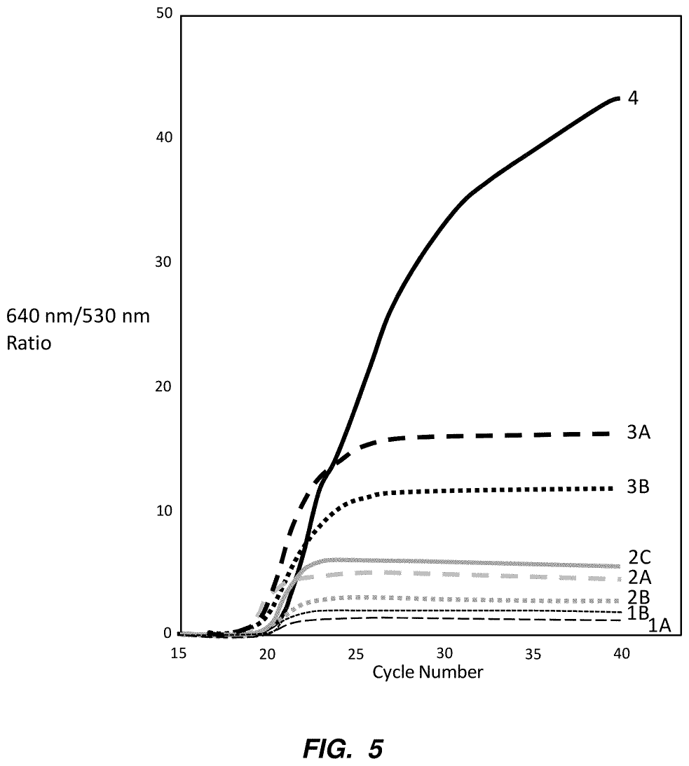

Signal-to-noise (S/N) ratio at each PCR cycle was calculated by dividing the fluorescent intensities at 640 nm by those at 530 nm. As shown in FIG. 5, the S/N ratio for the most part stabilized at cycle 25 at values that correlated to the number of acceptor ROX fluorophores on the double-stranded amplicon, i.e. the higher the number of acceptor fluorophores, the higher the S/N ratio. For clarity, Table 5 shows the S/N ratios for zero to four ROX copies at PCR cycles 25, 30 and 40. Where there were multiple primer configurations, the mean was shown. Compared to the single-labeled iFRET configuration (1 copy), the S/N ratio increased by 3-fold with two ROX copies, 9-fold with three ROX copies, and 12-fold and greater with four ROX copies, successfully increasing the reporter signal over the background signal of donor dye.

TABLE 5
Fluorescence S/N ratio (640 nm/530 nm)

| | |
|---|---|
| PCR cycle | Number of ROX label on the short amplicon |

| number | zero | 1 copy | 2 copies | 3 copies | 4 copies |
|---|---|---|---|---|---|
| 25 | 0.03 | 1.49 | 4.76 | 13.5 | 17.0 |
| 30 | 0.03 | 1.56 | 4.29 | 12.9 | 32.0 |
| 40 | 0.03 | 1.40 | 4.12 | 12.8 | 45.0 |

While this disclosure has been described using certain embodiments, it can be further modified while keeping within its spirit and scope. This application is therefore intended to cover any variations, uses, or adaptations of the disclosure using its general principles. This application is intended to cover any and all such departures from the present disclosure as come within known or customary practices in the art to which it pertains, and which fall within the limits of the appended claims.

### SEQUENCE LISTING

The instant application contains a Sequence Listing which has been submitted electronically as an XML file and is hereby incorporated by reference in its entirety. The electronically submitted XML file is named: “IMI-0004.NP Sequence Listing.xml”, was created on Dec. 13, 2022 and is 12,397 bytes in size.

What is claimed is:

1. A process for increasing the signal of a target-specific reporter molecule by induced fluorescence resonance energy transfer; the process comprising:
forming a reaction mixture by
providing a target-specific reporter molecule in a solution,
providing a sample of interest that may contain a target region in the solution, and
providing a double-stranded DNA-binding dye in the solution;
conducting an amplification reaction on the reaction mixture such that when the sample of interest contains the target region, the double-stranded DNA-binding dye binds to a target-reporter duplex formed between the target-specific reporter molecule and the target region, such that the target-reporter duplex has plural copies of a fluorophore covalently attached thereto;
illuminating the reaction mixture at a wavelength that excites the double-stranded DNA binding dye, such that fluorescence resonance energy transfer occurs from the double-stranded DNA binding dye to the fluorophores on the target-reporter duplex; and
detecting florescence of the fluorophores on the target-reporter duplex.

2. The process of claim 1, wherein providing the target-specific reporter molecule comprises providing a target-specific reporter molecule with plural copies of a fluorophore covalently attached thereto in a solution.

3. The process of claim 2, wherein providing the target-specific reporter molecule with plural copies of a fluorophore covalently attached thereto further comprises covalently attaching the plural copies of a fluorophore to a target-specific reporter molecule.

4. The process of claim 2, wherein the target specific reporter molecule comprises a first probe and further comprising
providing a second probe with plural copies of a second fluorophore covalently attached thereto in the solution, wherein the sample of interest may contain a second target region in the solution such that when conducting the amplification reaction on the reaction mixture when the sample of interest contains the second target region, the double-stranded DNA-binding dye binds to a second probe-target duplex formed between the second probe with plural copes of the second fluorophore and the second target region;
illuminating the reaction mixture at an excitation wavelength of the double-stranded DNA binding dye, such that fluorescence resonance energy transfer occurs from the double-stranded DNA binding dye to the second fluorophores on the second probe that forms the second target-reporter duplex; and
detecting florescence of the second fluorophores.

5. The process of claim 2, wherein providing the target-specific reporter molecule with plural copies of a fluorophore covalently attached thereto comprises providing a probe having a first copy of the fluorophore covalently attached to the 5′-end and a second copy of the fluorophore covalently attached to the 3′-end.

6. The process of claim 2, wherein providing the target-specific reporter molecule with plural copies of a fluorophore covalently attached thereto comprises providing an oligonucleotide having at least one copy of the fluorophore covalently attached to the 5′-end or the 3′ end and at least one additional copy of the fluorophore covalently attached to an internal portion through a linker.

7. The process of claim 6, wherein providing the target-specific reporter molecule with plural copies of a fluorophore covalently attached thereto comprises providing a probe having a first copy of the fluorophore covalently attached to the 5′-end, a second copy of the fluorophore covalently attached to the 3′-end and at least one additional copy of the fluorophore covalently attached to an internal portion through a linker.

8. The process of claim 2, wherein the target-specific reporter molecule includes multiple copies of the fluorophore attached to the internal portion through linkers.

9. The process of claim 1, wherein providing the target-specific reporter molecule comprises providing a first reporter molecule specific to a first region of a first target, and a second reporter molecule specific to a second region of a first target.

10. The process of claim 9, wherein the first reporter molecule is labeled with a first copy of the fluorophore, and the second reporter molecule is labeled with at least a second copy of the fluorophore, such that during amplification the first reporter molecule and the second reporter molecule form the single target-reporter duplex.

11. The process of claim 9, wherein the first and second reporter molecules are probes and have equivalent melting temperatures.

12. The process of claim 9, further comprising wherein providing a third reporter molecule specific to a second target.

13. The process of claim 12, wherein the first reporter molecule and the second reporter molecule are labeled with a plurality of a first fluorophore, and the third reporter molecule is labeled with a plurality of a second fluorophore.

14. The process of claim 1, wherein providing the target-specific reporter molecule comprises providing a first reporter molecule specific to first target, and a second reporter molecule specific to a second target.

15. The process of claim 14, wherein the first reporter molecule is labeled with a plurality of a first fluorophore, and the second reporter molecule is labeled with a plurality of a second fluorophore.

16. The process of claim 14, further comprising providing a third reporter molecule specific to a third target.

17. The process of claim 1, wherein the double-stranded DNA-binding dye binds to a target-reporter duplex formed between the target-specific reporter molecule and the target region, such that the target-reporter duplex has plural copies of a fluorophore covalently attached thereto by incorporating multiple acceptor fluorophores into the amplified target by use of labeled primers or by use of labeled deoxynucleotide triphosphate (dNTP) in the solution.

18. The process of claim 17, wherein incorporating multiple acceptor fluorophores into the amplified target by use of labeled primers comprises the use of a labeled forward primer and a labeled reverse labeled primer.

19. A reaction mixture which during PCR comprises:
a double-stranded DNA-binding dye;
a duplex formed by hybridization of reporter and target nucleic acids;
said duplex having a plurality of a fluorophore covalently attached thereto;
wherein the double-stranded DNA-binding dye is a donor and the fluorophore is an acceptor forming an iFRET relationship.

20. The reaction mixture of claim 19, wherein the target nucleic acids include a target-specific reporter molecule with plural copies of a fluorophore covalently attached thereto.


SEQUENCE LISTING (13 sequences)

SEQ ID NO: 1 - DNA, 17 bp, synthetic construct
  source 1..17: mol_type = other DNA
tccagggtgcatatgag

SEQ ID NO: 2 - DNA, 17 bp, synthetic construct
  source 1..17: mol_type = other DNA
ttagtaccgacctgacg

SEQ ID NO: 3 - DNA, 23 bp, synthetic construct
  source 1..23: mol_type = other DNA
aagagtttcttcctatgagagcc

SEQ ID NO: 4 - DNA, 19 bp, synthetic construct
  source 1..19: mol_type = other DNA
aaggctctgcgcggacttg

SEQ ID NO: 5 - DNA, 22 bp, synthetic construct
  source 1..22: mol_type = other DNA
ttcggaagagacaggtacgtta

SEQ ID NO: 6 - DNA, 19 bp, synthetic construct
  source 1..19: mol_type = other DNA
tattgcagcagtacgcaca

SEQ ID NO: 7 - DNA, 19 bp, synthetic construct
  source 1..19: mol_type = other DNA
actagccatccttactgca

SEQ ID NO: 8 - DNA, 19 bp, synthetic construct
  source 1..19: mol_type = other DNA
  misc_feature 13
  misc_feature 18
gcggtgtttgcanatttng

SEQ ID NO: 9 - DNA, 17 bp, synthetic construct
  source 1..17: mol_type = other DNA
ggctgtctccacaagtc

SEQ ID NO: 10 - DNA, 18 bp, synthetic construct
  source 1..18: mol_type = other DNA
aaggctctgcgcggactt

SEQ ID NO: 11 - DNA, 17 bp, synthetic construct
  source 1..17: mol_type = other DNA
ttaaaccaggtggaacc

SEQ ID NO: 12 - DNA, 16 bp, synthetic construct
  source 1..16: mol_type = other DNA
agttgtggcatctcct

SEQ ID NO: 13 - DNA, 38 bp, synthetic construct
  source 1..38: mol_type = other DNA
ttaaaccaggtggaacctcatcaggagatgccacaact